(19) United States
(12) Patent Application Publication
(10) Pub. No.: US 2024/0425786 A1
(43) Pub. Date: Dec. 26, 2024

(54) MULTIFUNCTIONAL BAG PORT AND MATING RING FOR A BIOPROCESSING SYSTEM

(71) Applicant: GLOBAL LIFE SCIENCES SOLUTIONS USA LLC, Marlborough, MA (US)

(72) Inventor: RICHARD DAMREN, MARLBOROUGH, MA (US)

(73) Assignee: GLOBAL LIFE SCIENCES SOLUTIONS USA LLC, MARLBOROUGH, MA (US)

(21) Appl. No.: 18/213,361

(22) Filed: Jun. 23, 2023

(51) Int. Cl. C12M 1/00; C12M 1/12; C12M 3/00

(52) U.S. Cl. CPC C12M 23/14; C12M 23/28; C12M 23/40; C12M 23/48; C12M 37/04

(57) ABSTRACT

A bioprocessing system for use in carrying out a biomanufacturing process is provided. The system includes a single-use bag configured to carry out a bioprocessing operation with at least one port welded to the single-use bag. The at least one port has a channel for providing fluidic access to an interior volume of the single-use bag. The system further includes at least one mating ring. The at least one port includes a first feature configured to engage with a corresponding second feature of the at least one mating ring, such that the at least one mating ring can be locked to the at least one port. The mating ring and bag port are configured to provide an array of structural functionalities to aid the assembly and use of the bioprocessing system.

### TECHNICAL FIELD

Embodiments of the invention relate generally to bioprocessing systems and methods and, more particularly, to modified bag ports on a single-use bioprocessing bag and associated mating rings.

### BACKGROUND

A variety of vessels, devices, components and unit operations are known for carrying out biochemical and/or biological processes and/or manipulating liquids and other products of such processes. In order to avoid the time, expense, and difficulties associated with sterilizing the vessels used in biopharmaceutical manufacturing processes, single-use or disposable bioreactor bags and single-use mixer bags are used as such vessels. For instance, biological materials (e.g., animal and plant cells) including, for example, mammalian, plant or insect cells and microbial cultures can be processed using disposable or single-use mixers and bioreactors.

Increasingly, in the biopharmaceutical industry, single use or disposable containers are used. Such containers can be flexible or collapsible plastic bags that are supported by an outer rigid structure such as a stainless-steel shell or vessel. Use of sterilized disposable bags eliminates the time-consuming step of cleaning of the vessel and reduces the chance of contamination. The bag may be positioned within the rigid vessel and filled with the desired fluid for mixing. Depending on the fluid being processed, the system may include a number of fluid lines and different sensors, probes and ports coupled with the bag for monitoring, analytics, sampling, and fluid transfer. For example, a plurality of ports may typically be located at the front of the bag and accessible through an opening in the sidewall of the vessel, which provide connection points for sensors, probes and/or fluid sampling lines.

According to the state of the art as illustrated by FIG. 1, existing bioprocessing systems 1 include an outer vessel 2 that houses a single-use bag 3. The bag 3 includes ports 4 located at an opening in the vessel 2 that have standard barbs which are connected to tubes, with no additional functionality. In other words, the ports 4 provide a single function—connection to tubing such that the internal volume of the bag is sterilely accessible. However, when multiple ports 4 are used, a port plate 5 is often implemented to ensure that the ports 4 are aligned within the opening in the sidewall of the vessel and to support the bag in the unsupported area of the opening in the sidewall of the vessel. Installation of such a port plate 5 is cumbersome, and often difficult due at least in part to the fact that the port plate 5 cannot be fixed to the bag 3.

Additionally, the ports are welded to the bag, which provide a unique opportunity to add structural functionality directly attached to the bag, which the art has yet to envisage. For example, instead of using separate probe support structures as is standard in the art today, additional functionality could allow the probes to be directly supported by the bag port.

In view of the above, the present invention provides a means to add multiple structural functions to bag ports, and well as a means to aid in the installation of bioprocessing components.

### BRIEF DESCRIPTION

One aspect of the invention relates to a bioprocessing system, including a single-use bag configured to carry out a bioprocessing operation; at least one port welded to the single-use bag, the at least one port including a channel for providing fluidic access to an interior volume of the single-use bag; and at least one mating ring; wherein the at least one port includes a first feature configured to engage with a corresponding second feature of the at least one mating ring, such that the at least one mating ring can be locked to the at least one port.

In embodiments, the first feature comprises at least one groove and the second feature comprises at least one protrusion, and the at least one mating ring is locked to the at least one port by inserting the at least one protrusion into the at least one groove and rotating the at least one mating ring relative to the at least one port. In further embodiments, the first feature comprises at least one snap lock and the second feature comprises at least one protrusion, and the at least one mating ring is locked to the at least one port by inserting the at least one protrusion into the at least one snap lock.

In embodiments, the system further includes a port plate comprising at least one opening, the at least one opening corresponding to a location of the at least one port. The at least one opening is aligned with the at least one port and the at least one mating ring is locked to the at least one port, the port plate is secured to the single-use bag. The single-use bag is configured to be placed in an outer vessel, the outer vessel having an opening corresponding to the location of the at least one port, and the at least one mating ring includes an alignment feature, the alignment feature configured to align the port plate with the opening.

In embodiments, the at least one mating ring further comprises a probe support, the probe support configured to physically support a probe attached to the at least one port. The probe support is configured to maintain the probe at an angle relative to a longitudinal axis of the channel.

In embodiments, the system further includes a protective cover configured to cover the at least one port during shipping, and when the cover is aligned with the at least one port and the at least one mating ring is locked to the at least one port, the cover is secured to the single-use bag.

In embodiments, the system further includes at least one mating ring further comprises an attachment mechanism configured to be attached to a hook or clip, and when the hook or clip is attached, the hook or clip is configured to further attach to an external structure.

In embodiments, the at least one port comprising a plurality of ports and the at least one mating ring comprises a plurality of mating rings, and each of the plurality of mating rings is configured to lock to a corresponding port of the plurality of ports.

Another aspect of the invention relates to a method for providing a locking feature on a single-use bioprocessing bag. The method includes welding at least one port to the single-use bioprocessing bag, the at least one port including a channel for providing fluidic access to an interior volume of the single-use bioprocessing bag, wherein the port includes a first feature; providing at least one mating ring, the at least one mating ring having a second feature; and engaging the first feature with the second feature such that the at least one mating ring is locked to the at least one port.

In embodiments, the first feature comprises at least one groove and the second feature comprises at least one protrusion, and engaging the first feature with the second feature comprises inserting the at least one protrusion into the at least one groove and rotating the at least one mating ring relative to the at least one port.

In further embodiments, the first feature comprises at least one snap lock and the second feature comprises at least one protrusion, and engaging the first feature with the second feature comprises inserting the at least one protrusion into the at least one snap lock.

In embodiments, the method further includes aligning a port plate with the at least one port prior to engaging the feature with the second feature, such that when the at least one mating ring is locked to the at least one port, the port plate is secured to the single-use bioprocessing bag. The single-use bag is configured to be placed in an outer vessel, the outer vessel having an opening corresponding to the location of the at least one port, and the at least one mating ring includes an alignment feature, the alignment feature configured to align the port plate with the opening.

In embodiments, the at least one mating ring further comprises a probe support, the probe support configured to physically support a probe attached to the at least one port, and the probe support maintains the probe at an angle relative to a longitudinal axis of the channel.

In embodiments, the method further includes attaching a protective cover to he at least one port, and when the cover is aligned with the at least one port and the at least one mating ring is locked to the at least one port, the cover is secured to the single-use bag.

In embodiments, the at least one mating ring further comprises an attachment mechanism, and attaching a hook or clip to the attachment mechanism, the hook or clip configured to further attach to an external structure.

### DRAWINGS

The present invention will be better understood from reading the following description of non-limiting embodiments, with reference to the attached drawings, wherein below:

FIG. 1 is a perspective view of a bioprocessing system according to the state of the art.

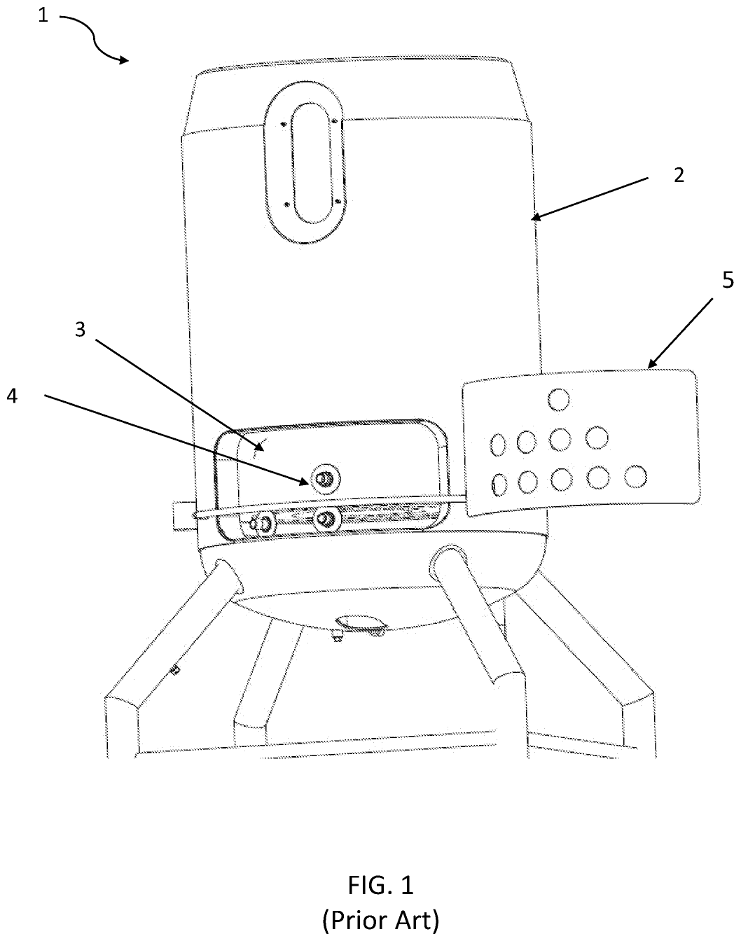

FIG. 2 is a perspective view of a bioprocessing system implementing the modified bag port and attachment mechanism, according to an embodiment of the present invention.

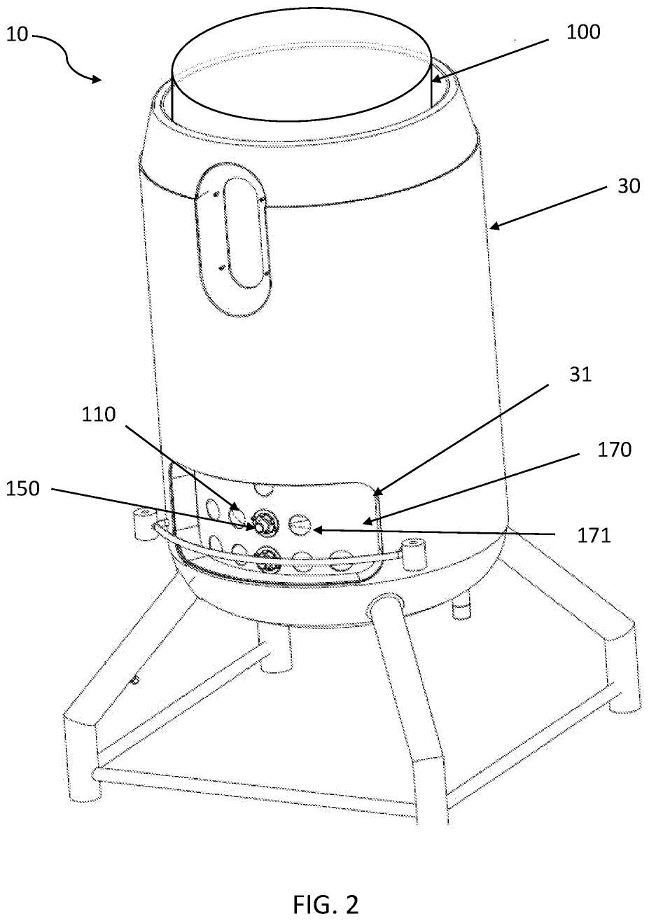

FIGS. 3A and 3B illustrate exploded and assembled views of a modified bag port and mating ring, according to embodiments of the invention.

FIGS. 4A and 4B illustrate exploded and assembled views of a modified bag port and mating ring, according to further embodiments of the invention.

FIGS. 5A and 5B illustrate exploded and assembled views of a modified bag port and mating ring, according to additional embodiments of the invention.

FIG. 6 illustrates the modified port and mating ring used to align and/or secure a port plate to a single-use bag, according to embodiments of the invention.

FIGS. 7A and 7B illustrate exploded and assembled views of a modified bag port and mating ring having an alignment feature, according to embodiments of the invention.

FIGS. 8A and 8B illustrate the modified port and mating ring with alignment mechanism used to align the ports and port plate within a vessel opening, according to embodiments of the invention.

FIGS. 9A and 9B illustrate exploded and assembled views of a modified bag port and mating ring having a probe support, according to embodiments of the invention.

FIGS. 10A and 10B illustrate exploded and assembled views of a modified bag port and mating ring having a probe support, according to further embodiments of the invention.

FIGS. 11A and 11B illustrate exploded and assembled views of a modified bag port and mating ring having an attachment mechanism, according to further embodiments of the invention.

FIGS. 12A and 12B illustrate exploded and assembled views of the modified port plate and mating ring to secure a protective cover for packaging and/or shipping, according to embodiments of the invention.

### DETAILED DESCRIPTION

Reference will be made below in detail to exemplary embodiments of the invention, examples of which are illustrated in the accompanying drawings. Wherever possible, the same reference characters used throughout the drawings refer to the same or like parts.

As used herein, the term “flexible” or “collapsible” refers to a structure or material that is pliable, or capable of being bent without breaking, and may also refer to a material that is compressible or expandable. An example of a flexible structure is a bag formed of polyethylene film. The terms “rigid” and “semi-rigid” are used herein interchangeably to describe structures that are “non-collapsible,” that is to say structures that do not fold, collapse, or otherwise deform under normal forces to substantially reduce their elongate dimension. Depending on the context, “semi-rigid” can also denote a structure that is more flexible than a “rigid” element, e.g., a bendable tube or conduit, but still one that does not collapse longitudinally under normal conditions and forces.

A “vessel,” as the term is used herein, means a flexible bag, a flexible container, a semi-rigid container, a rigid container, or a flexible or semi-rigid tubing, as the case may be. The term “vessel” as used herein is intended to encompass bioreactor vessels having a wall or a portion of a wall that is flexible or semi-rigid, single use flexible bags, as well as other containers or conduits commonly used in biological or biochemical processing, including, for example, cell culture/purification systems, mixing systems, media/buffer preparation systems, and filtration/purification systems, e.g., chromatography and tangential flow filter systems, and their associated flow paths. As used herein, the term “bag” means a flexible or semi-rigid container or vessel used, for example, as a bioreactor or mixer for the contents within. As used herein, “consumable” or “consumable component” means devices or components that are intended to be replaced regularly due to wear or use.

Embodiments of the invention provide bioprocessing systems and, in particular, a bioprocessing system 10 (hereafter referred to as “bioprocessing system” or “system”). The system 10 includes an outer vessel 30 that is configured to house a single-use bag 100. The bag 100 includes at least one port 110 which is configured to be located in at an opening 31 in the outer vessel 30. As will be described in greater detail below, port 110 is modified such that it can engage with a mating ring 150, thereby allowing the mating ring 150 to be locked to the at least one port 110. In this way, when openings 171 in the port plate 170 are aligned with the at least one port 110, mating ring(s) 150 can be connected to the ports 110, fixing the port plate 170 in place during use. Moreover, additional modifications to the at least one port 100, as further described below, provide additional functionality that has yet to be seen in the art.

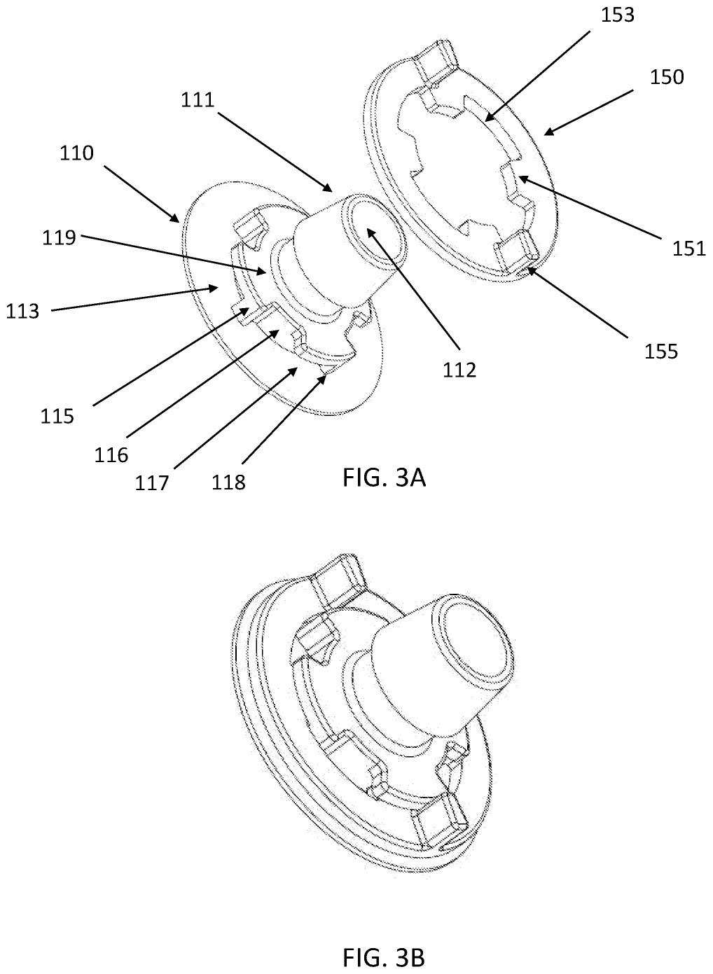

FIGS. 3A and 3B illustrate an assembled (FIG. 3B) and exploded view (FIG. 3A) of a modified port 110 and mating ring 150, according to embodiments of the invention. The port 110 includes a proximal end formed as a barb 111 having a channel 112 therein. The channel 112 provides fluidic access to an interior volume of the single-use bag 100. The distal end is wended to the single-use bag 100 and includes a body portion 113. The body 113 includes a first feature that is configured to engage with the mating ring 150. In the illustrated embodiment, the first feature is a twist lock 115 that comprises at least one opening 116 and groove 117 formed in the body 113. The mating ring 150 has a generally flat, ring shape. The mating ring 150 includes a second feature in the form of at least one protrusion 151, extending from the circumference of the ring shape towards the center of the mating ring 150.

To lock the port 110 to the mating ring 150, the protrusion(s) 151 are inserted into the opening(s) 116, and the mating ring 150 is rotated (e.g., counterclockwise) such that the protrusion(s) 151 travel through the groove(s) 117 until they abut side walls 118 of the body 113. Rotation can be facilitated by tabs 155 (e.g., a user can rotate the mating ring 150 by grasping and rotating tabs 155). Once the protrusion(s) 151 are abutting the side wall(s) 118, the mating ring 150 is prevented from being separated (e.g., is locked) from the port 110 due to the top surface 119 of the body 113 extending over the groove(s) 117 (see, e.g., FIG. 3B).

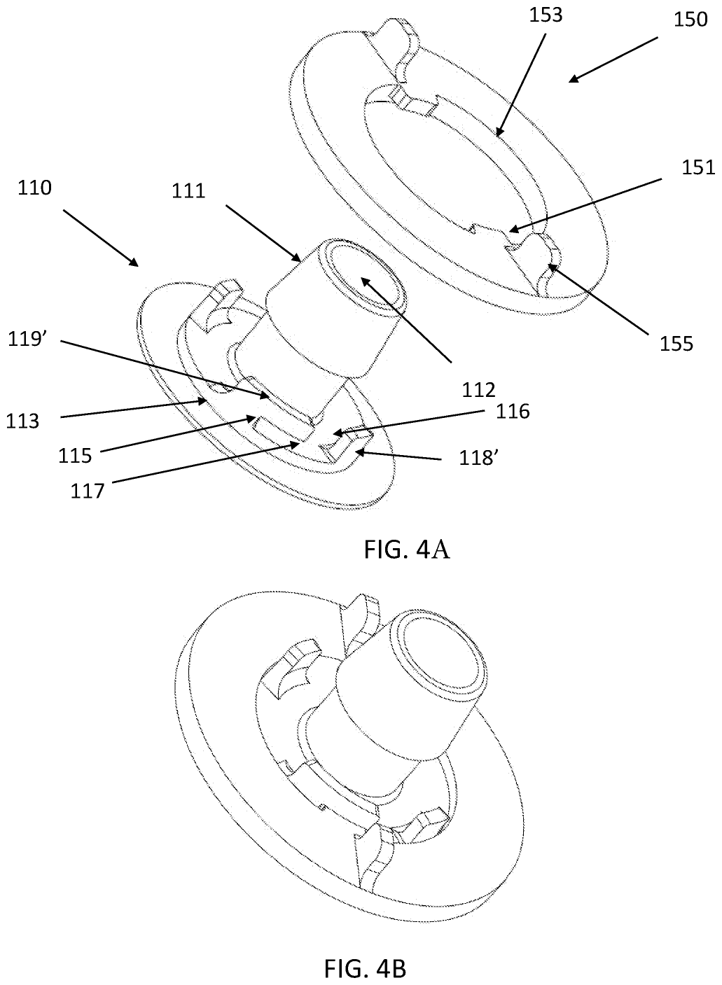

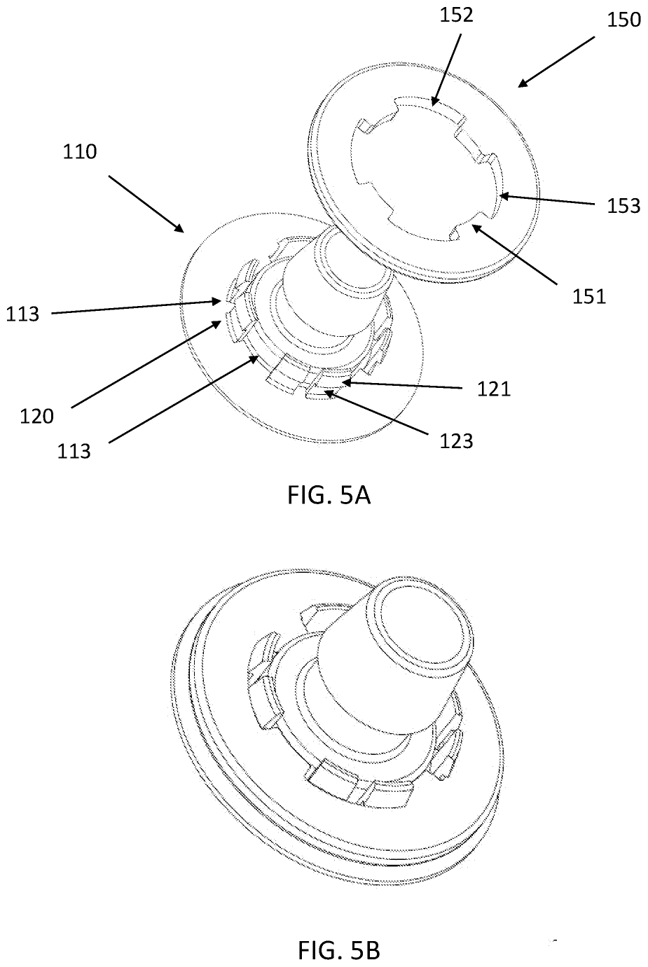

FIGS. 4A and 4B illustrate an assembled (FIG. 5B) and exploded view (FIG. 5A) of a modified port 110 and mating ring 150, according to further embodiments of the invention. The embodiment of FIGS. 4A and 4B is similar to that of FIGS. 3A and 3B, but the body 113 does not include a planar top surface 119. Rather, the opening(s) 116 and groove(s) 117 are formed by side-wall sections 118′ (e.g., in the form of upstanding protrusions) and lateral projections 119′. The other features of the embodiment of FIGS. 4A and 4B are the same or substantially the same as that of FIGS. 3A and 3B, the disclosure of which is omitted for conciseness.

Additionally, the protrusions 151 and/or twist lock 115 can further include additional locking mechanisms that prevent the mating ring 150 from being rotated in the opposite direction (e.g., clockwise) and unlocked from the port 110. The additional locking mechanisms could be in the form of mechanical locks, such as snap fit engagements, magnetic locks, such as providing magnets of opposing polarity in each of the port 110 and mating ring 150, or other comparable securing means (e.g., a user could glue the port and mating ring together once they are engaged with each other. However, it is explicitly noted that additional locking mechanisms are not required in order to sufficiently lock the mating ring 150 to the port 110 for the purposes of the present invention. Specifically, the twist lock 115 and protrusion(s) 151 provide sufficient mechanical restraint to provide the improved functions as described herein.

FIGS. 5A and 5B illustrate an assembled (FIG. 5B) and exploded view (FIG. 5A) of a modified port 110 and mating ring 150, according to still further embodiments of the invention. Rather than implementing a twist lock feature 115 for securing the port 110 to the mating ring 150, this embodiment implements a snap lock 120. In particular, the body 130 comprises a first feature that includes at least one snap lock 120 around its periphery. Each snap lock 120 includes a tab 121 and groove 123. The geometry of the snap lock(s) 120 is such that the inner side wall 152 of the mating ring can be snapped into the snap lock(s) 120. In particular embodiments, the snap lock(s) 120 can be spaced apart from one another around the periphery of the body 113, such that the protrusions 151 fit into the spaces between snap lock(s) 120, which helps ensure that the mating ring cannot rotate one it is locked to the port 110. In addition, these spacings advantageously can be used as alignment guides for the bag port during the port welding process. The barbed bag port welding fixturing could have complementary features located in the fixturing alignment plates that interacts with these bag port alignment features to define the port weld orientation on bag. Additionally, features on the bag film panel could be used to align the panel with the port welding fixturing, and bag panel features such as other existing bag port openings, or special alignment holes could serve to align the bag film panel.

To lock the port 110 to the mating ring 150, a user presses the mating ring against the port 110. In doing so, the snap lock(s) 120 flex, allowing the mating ring 150 to pass through and sit within the groove 123. Since the groove(s) 123 are recessed from the tab(s) 121, the tab(s) are allowed to flex back to their original position (see, e.g., FIG. 5B) such that they retain the mating ring 150 in the groove(s) 123.

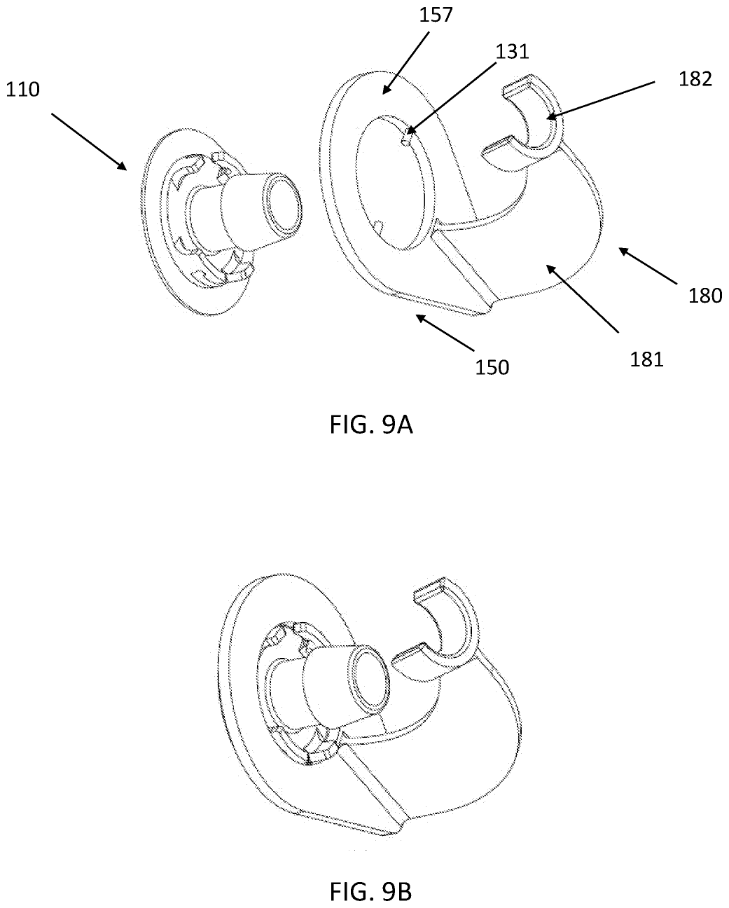

While the above-described embodiments illustrate specific examples of modified ports 110 and mating rings 150, the invention is not limited. For example, the number of protrusions 151, twist locks 115 and snap locks 120 can be varied. Further, the tabs 155 can be implement or omitted in any embodiment of the invention, and the protrusions can take the form of pins 131 (see, e.g., FIG. 9A). Even further, other mechanisms to attach the mating ring 150 to the port 110 are contemplated. For example, rather than using mechanical fixation, magnetics can be located on (or embedded in) the port 110 and mating ring 150. By placing magnets of opposing polarity in the port 110 and mating ring 150, the port 110 and mating ring 150 can be magnetically coupled and thereby locked to one another.

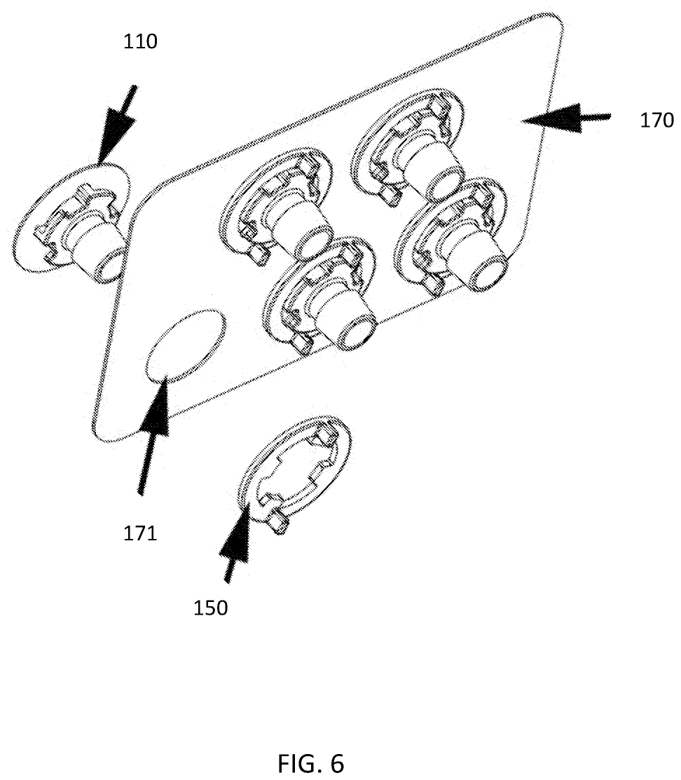

As mentioned above, and with reference to FIG. 6, alignment of port plate 170 in the opening 32 of the vessel is often difficult, as the port plate has no way of being fixed to the bioprocessing system 10. Particularly and according to the state of the art, the probe port plate is not tightly positioned/located around the barbed bag ports. Some clearance access is required close to the probe plate surface during the part of the bag assembly process when tubing, probe adapters, etc. are attached to those bag ports. Thus, the probe port plate is free to move with respect to the bag ports even after the probe adapters and other tubing have been attached to the ports. Cable ties on the tubing attached to the probe ports provides some degree of restraint but is insufficient to fully restrain movement. Often a cable tie can pass through the opening in the plate between the port tubing and the opening and moves in between the bag and the port plate. This holds the probe port and bag away from the probe port plate on the side wall of the bag, creating a pocket, which causes wrinkles, thus creating unwanted stress on the bag in that area. However, the modified port 110 and mating ring 150 of the present invention provide an easier and more advantageous way of aligning the port plate 170. As illustrated, when the port plate 170 is to be attached to bioprocessing system, a user first aligns the openings 171 of the port plate 170 with the ports 110. After alignment, mating rings 150 can be attached to the ports 110 (as described above), thereby locking and securing the port plate 170 to the single-use bag 100. Without such a locking mechanism the port plate 170 is free to float around and become misaligned with the ports 110.

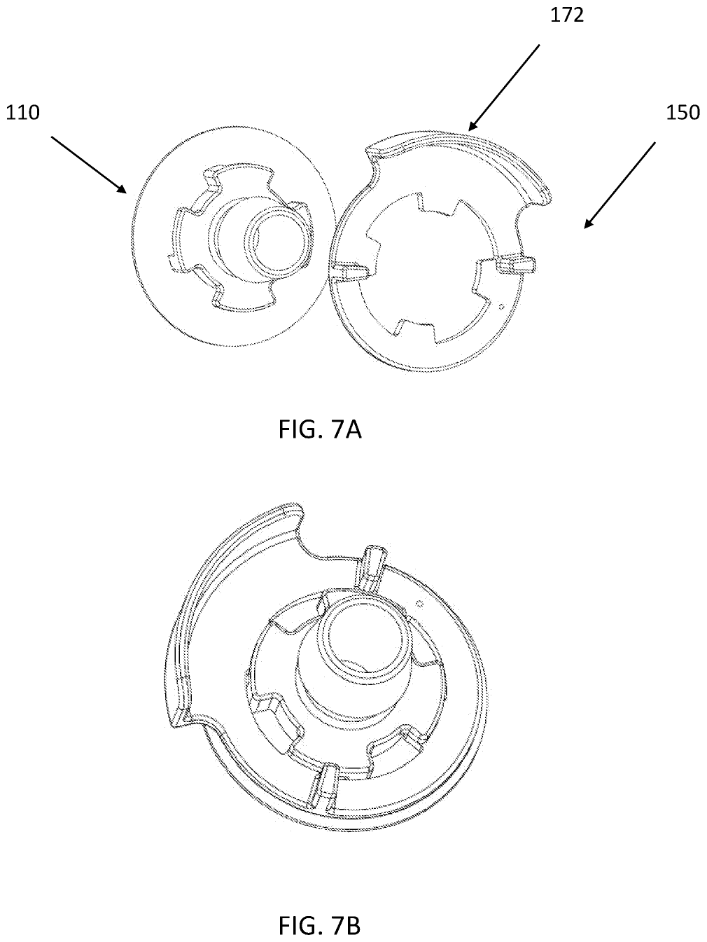

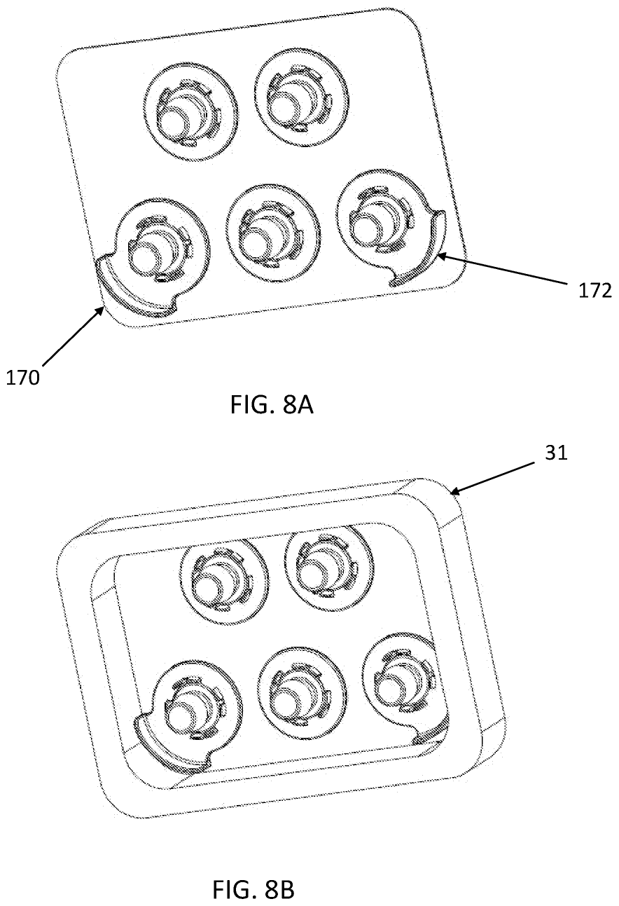

According to further embodiments, and as illustrated by FIGS. 7A and 7B, the mating ring 150 can include at least one alignment feature 172. The alignment feature 172 can be in the form of an arcuate side wall that extends around a portion of the periphery of the mating ring 150. As FIGS. 8A and 8B illustrate, the alignment features 172 can be located on mating rings 150 that are adjacent to the periphery of the opening 31 in the vessel 30. In this way, alignment features 172 further aides in aligning the port plate 170 with the opening 31. More specifically, there is not presently a mechanism on the bag and/or probe plate assembly to tightly control the position of the probe port plate with respect to the vessel opening. The bag ports in the region of the probe port plate are one of the main areas of interfacing between the user and the single-use bag. This interface would be improved if the probe port plate could be easily, accurately, and repeatably positioned with respect to the vessel opening. State of the art bag installation processes require the user to manually maintain this alignment during the bag install by using visual markings on the probe port plate to align with the vessel alcove opening. It would be desirable to make this alignment more accurate and have the alignment accuracy less dependent on manual intervention by the user, which is realized by the present inventions implementation of the aforementioned port 110 and mating ring 150 with associated alignment feature(s) 172.

In addition to providing means for securing a port plate 170, the mating ring 150 can include additional features to support further functionality. In embodiments illustrated in FIGS. 9A and 9B, the mating ring can include a probe support 180 configured to physically support a probe attached to the port 110. The probe support 180 can include an arm 181 extending away from a proximal face 157 of the mating ring 150. The arm 181 terminates at a cradle 182. The geometry of the arm 181 and cradle 182 is such that when a probe is attached to the port 110, the probe is maintained at a specific angle relative to a longitudinal axis of the channel 112 of the port 110. In embodiments, the angle is between 5-30 degrees. In one preferred embodiment, the angle is 15 degrees. Compared to this and according to the state of the art, users install the bag in the outer vessel and manually align markings on the probe port plate with the vessel alcove opening. A probe support bar which is mounted to the vessel is then manually adjusted to bring it up to a position under the probe body such that the probes are moved to a recommended angle (e.g., 15 degrees). However, the angle is set by eye by the user, the probe support bar can only be set at one of several discrete height settings by the user and the probe port plate on the bag itself is manually located by the user. All these manual adjustments lead to inaccuracies in the final setting of the sensor angle. In contrast, the present probe support 180 avoids all of these manual operations. Particularly, the present invention provides features that allow for consistent and repeatable installation of probes on the single-use bag 100.

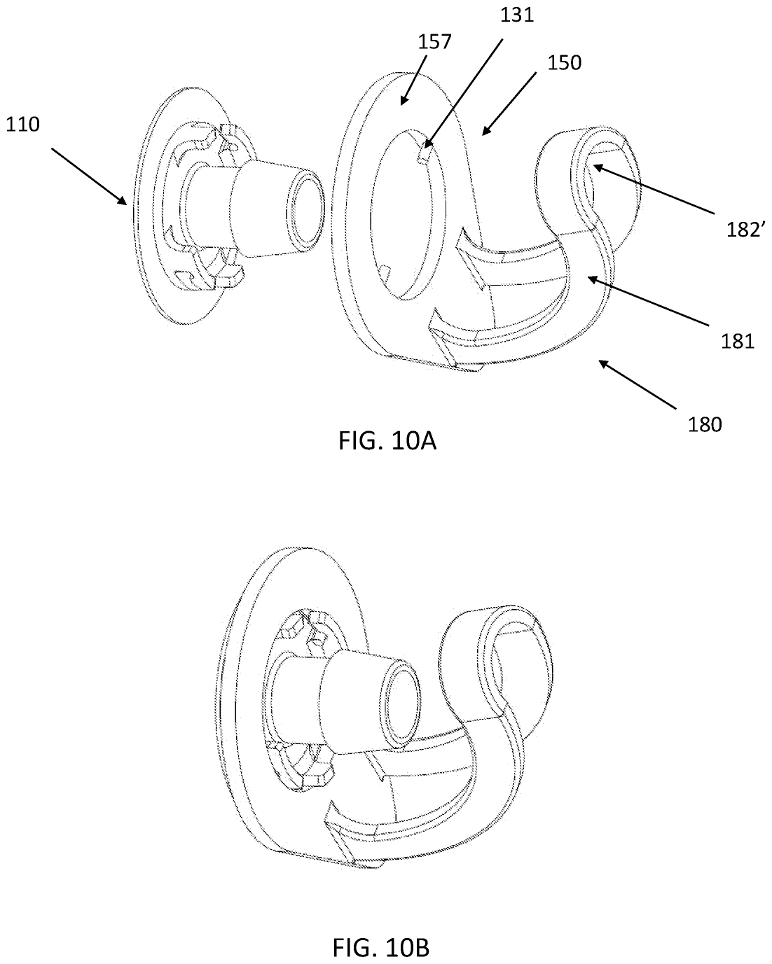

FIGS. 10A and 10B illustrate a variation of the embodiment of FIGS. 9A and 9B. As illustrated, the angle guide 180 can include an arm 181 that is attached to the proximal surface 157 at two locations. The arm 181 extends outwardly away from the proximal surface 157, bends in order to create a cradle 182′ and further extends back towards and attaches to the proximal surface 157. In this embodiment, angle guide 180 acts as to limit the maximum probe angle by preventing the probe from being raised more than 5-30 degrees. In one preferred embodiment, the angle is 15 degrees.

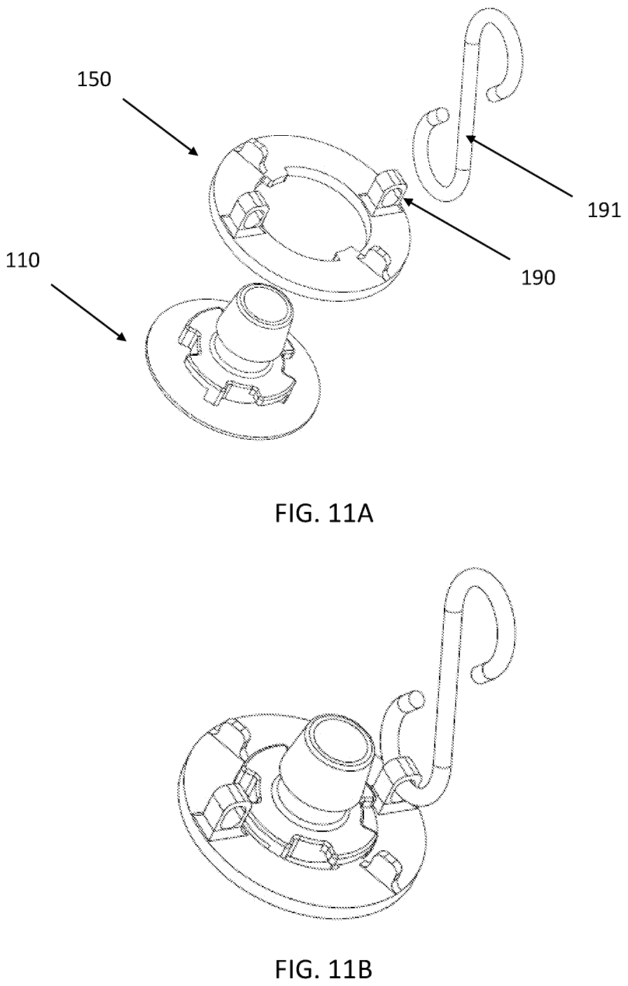

In addition to providing the above functions, the mating ring 150 can further include an attachment mechanism 190, as illustrated by FIGS. 11A and 11B. The attachment mechanism 190 is configured to be secured to a hook or clip 191 (see, e.g., FIG. 11B). The hook or clip 191 can then be attached to an external structure. In this way, ports 110 located on the top of the single-use bag 100 can be used as an anchor or support. For example, according to the state of the art, tubing connected to bag ports at the top of the bag are draped over a tubing manager arms at the top of the bag to bring some level of organization to the tubing itself. The tubing may provide a secondary function of helping to support the top of the bag during bag install and inflation, but this is a cumbersome way to support the bag and using the tubing as a support increases the likelihood of compromising the integrity of the tubing. Also, using the tubing as the bag support mechanism allows the user to drape the tubing anywhere along the tubing length which affects the position (height) at which the bag is supported. Thus, the user could drape the tubing over the support bar at a tubing location far from the bag and thus the bag would be essentially unsupported during inflation. In contrast, the attachment mechanism 190 in conjunction with hook or clip 191 allows a user to connect the hook or clip 191 to the tubing manager arms (or other external structure), providing direct support to the bag during installation and inflation, avoiding the need to use the tubing itself as a physical support, thereby providing a more repeatable, secure, and safe means for securing the bag during inflation. Further, by obviating the need for the tubing to act as a support mechanism the tubing can be draped anywhere along their length.

Moreover, by implementing the attachment mechanism 190 and a hook or clip 191, the ports 110 at the top of the bag can be fixed at a specific location, aiding the attachment of sensors (or other components) to the port 110. Similarly, by fixing the location of the ports 110, structures internal to the single-use bag 100 can be provided in specific and desired orientations. For example, by orienting the single-use bag 100 and the top-mounted ports 110, internal tubes, orifices, spray nozzles, and the like can be accurately positioned such that they are specific locations within the single-use bag 100.

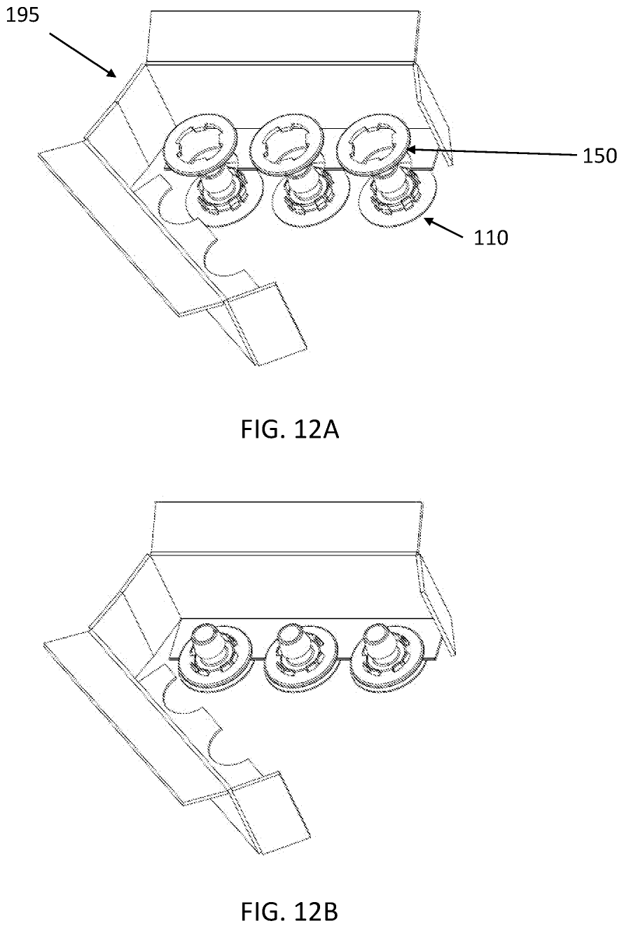

Still further to the above-described functions, and as illustrated by FIGS. 12A and 12B, the modified port(s) 110 and associated mating ring(s) 150 are configured to provide a means for securing a protective cover 195 to the single-use bag 100. As shown, a protective cover 195, in the form of a box, covers the ports 110 such that the mating rings 150 can be attached to the ports 110 after the box is aligned with the ports 110, thereby locking the protective cover to the ports 110 and the single-use bag 100. This is particularly advantageous when the single-use bag is required to be shipped. In particular and according to the state of the art, a completed bag assembly needs to be folded in such a way that the tubing attached to the bag ports can be packaged without damaging the folded bag assembly. This usually requires coiling the tubing, securing the tubing bundle with some sort of cable tie and covering any exposed cable tie ends or potentially sharp connector end caps with bubble wrap to keep them from damaging the bag during shipping and handling. Some of the exposed tubing and connectors are packaged in small bags. The sensor ports with their special sanitary fittings also require packaging that keeps them from damage and from them damaging the bag while the bag assembly is folded during shipping and handling. All this packaged tubing, connectors, etc. are carefully placed in the packaged bag assembly, but there is no easy way to ensure that all of these materials do not move during shipping and handling. This complicated packaging requires lots of bubble wrap, cable ties and other packaging materials to be removed from the packaged bag assembly by the user before installing the bag in the vessel. In contrast, by implementing the modified port(s) 110 and mating ring(s) 150, the protective cover 195 can be simply and easily installed, thereby protecting the ports, tubes, connectors, etc., without the need for the above complicated packaging. Moreover, such a protective cover 195 reduces the amount of packing materials required, thereby providing a more sustainable solution for shipping and handling.

It is explicitly noted that the any of the features described above can be combined such that any of the ports 110 welded to the single-use bag 100 can provide any or all of the aforementioned functions. For example, a mating ring 150 could include any combination of alignment features 170, probe supports 180, and attachment mechanisms 190. Similarly, the locking feature for any of the previously described embodiments can be in the form of the previously described twist lock 115, snap lock 120 or other comparable locking mechanisms.

It is noted that the above description describes modified ports 110 and associated mating rings 150 for providing additional functionality not presently seen in the art. The additional functionality provides numerous advantages over the prior art, including but not limited to, improved usability and installation of the single-use bag, easy customization, and reduced costs and complexity associated with shipping.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property.

This written description uses examples to disclose several embodiments of the invention, including the best mode, and also to enable one of ordinary skill in the art to practice the embodiments of invention, including making and using any devices or systems and performing any incorporated methods. The patentable scope of the invention is defined by the claims, and may include other examples that occur to one of ordinary skill in the art. Such other examples are intended to be within the scope of the claims if they have structural elements that do not differ from the literal language of the claims, or if they include equivalent structural elements with insubstantial differences from the literal languages of the claims.

1. A bioprocessing system, comprising:
a single-use bag configured to carry out a bioprocessing operation;
at least one port welded to the single-use bag, the at least one port including a channel for providing fluidic access to an interior volume of the single-use bag; and
at least one mating ring;
wherein the at least one port includes a first feature configured to engage with a corresponding second feature of the at least one mating ring, such that the at least one mating ring can be locked to the at least one port.

2. The bioprocessing system of claim 1,
wherein the first feature comprises at least one groove and the second feature comprises at least one protrusion, and
wherein the at least one mating ring is locked to the at least one port by inserting the at least one protrusion into the at least one groove and rotating the at least one mating ring relative to the at least one port.

3. The bioprocessing system of claim 1,
wherein the first feature comprises at least one snap lock and the second feature comprises at least one protrusion, and
wherein the at least one mating ring is locked to the at least one port by inserting the at least one protrusion into the at least one snap lock.

4. The bioprocessing system of claim 1, further comprising:
a port plate comprising at least one opening, the at least one opening corresponding to a location of the at least one port;
wherein when the at least one opening is aligned with the at least one port and the at least one mating ring is locked to the at least one port, the port plate is secured to the single-use bag.

5. The bioprocessing system of claim 4,
wherein the single-use bag is configured to be placed in an outer vessel, the outer vessel having an opening corresponding to a location of the at least one port, and
wherein the at least one mating ring includes an alignment feature, the alignment feature configured to align the port plate with the opening.

6. The bioprocessing system of claim 1,
wherein the at least one mating ring further comprises a probe support or angle guide, the probe support configured to physically support a probe attached to the at least one port.

7. The bioprocessing system of claim 6,
wherein the probe support or angle guide is configured to maintain the probe at an angle relative to a longitudinal axis of the channel.

8. The bioprocessing system of claim 1, further comprising a protective cover configured to cover the at least one port during shipping,
wherein when the cover is aligned with the at least one port and the at least one mating ring is locked to the at least one port, the cover is secured to the single-use bag.

9. The bioprocessing system of claim 1,
wherein the at least one mating ring further comprises an attachment mechanism configured to be attached to a hook or clip, and
wherein when the hook or clip is attached, the hook or clip is configured to further attach to an external structure.

10. The bioprocessing system of claim 1,
wherein the at least one port comprising a plurality of ports and the at least one mating ring comprises a plurality of mating rings, and
wherein each of the plurality of mating rings is configured to lock to a corresponding port of the plurality of ports.

11. A method for providing a locking feature on a single-use bioprocessing bag, the method comprising:
welding at least one port to the single-use bioprocessing bag, the at least one port including a channel for providing fluidic access to an interior volume of the single-use bioprocessing bag, wherein the port includes a first feature;
providing at least one mating ring, the at least one mating ring having a second feature; and
engaging the first feature with the second feature such that the at least one mating ring is locked to the at least one port.

12. The method of claim 11,
wherein the first feature comprises at least one groove and the second feature comprises at least one protrusion, and
wherein engaging the first feature with the second feature comprises inserting the at least one protrusion into the at least one groove and rotating the at least one mating ring relative to the at least one port.

13. The method of claim 11,
wherein the first feature comprises at least one snap lock and the second feature comprises at least one protrusion, and
wherein engaging the first feature with the second feature comprises inserting the at least one protrusion into the at least one snap lock.

14. The method of claim 11 further comprising:
aligning a port plate with the at least one port prior to engaging the feature with the second feature, such that when the at least one mating ring is locked to the at least one port, the port plate is secured to the single-use bioprocessing bag.

15. The method of claim 14, wherein the single-use bag is configured to be placed in an outer vessel, the outer vessel having an opening corresponding to the location of the at least one port, and
wherein the at least one mating ring includes an alignment feature, the alignment feature configured to align the port plate with the opening.

16. The method of claim 11,
wherein the at least one mating ring further comprises a probe support, the probe support configured to physically support a probe attached to the at least one port, and
wherein the probe support maintains the probe at an angle relative to a longitudinal axis of the channel.

17. The method of claim 11, the method further comprising:
attaching a protective cover to he at least one port,
wherein when the cover is aligned with the at least one port and the at least one mating ring is locked to the at least one port, the cover is secured to the single-use bag.

18. The method of claim 11,
wherein the at least one mating ring further comprises an attachment mechanism, and
attaching a hook or clip to the attachment mechanism, the hook or clip configured to further attach to an external structure.
